(19) United States
(12) Patent Application Publication
(10) Pub. No.: US 2004/0236151 A1
(43) Pub. Date: Nov. 25, 2004

(54) Process to produce enantiomerically enriched 1-aryl-and 1-heteroaryl-2-aminoethanols

(75) Inventors: Steven P. Tanis, Carlsbad, CA (US); James A. Nieman, Carlsbad, CA (US)

Correspondence Address: AGOURON PHARMACEUTICALS, INC., 10350 NORTH TORREY PINES ROAD, LA JOLLA, CA, 92037, US

(73) Assignee: Pfizer, Inc.

(21) Appl. No.: 10/804,296

(22) Filed: Mar. 19, 2004

Related U.S. Application Data
(60) Provisional application No. 60/457,793, filed on Mar. 26, 2003.

(51) Int. Cl. C 07C 2 9/06

(52) U.S. Cl. 564/483

(57) ABSTRACT

The invention relates to a method of preparing enantiomerically enriched amino alcohols of Formula I

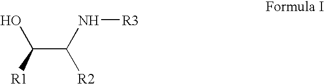

wherein the variable R1, R2, and R3 are defined herein.

This application claims priority under 35 U.S.C. 119(e) to U.S. Application No. 60/457,793, filed Mar. 26, 2003.

### BACKGROUND OF THE INVENTION

Amino alcohols are important compounds for use as pharmaceutical agents, intermediates for pharmaceutical agents, polymers, chelating agents, chiral auxiliaries and the like.

### SUMMARY OF THE INVENTION

This invention describes a convenient method for the preparation and use of a ruthenium catalyst for a chiral reduction of ketones. A further aspect of the invention is the preparation of amino alcohols, particularly chiral 1,2-amino alcohols. A number of syntheses of these important compounds have been described. Methods include for example, reduction of amino ketones, reduction of alpha-hydroxy amides, reaction of epoxides with amines, reaction of halohydrins with amines, reaction of an alpha-amino organo-lithium with an aldehyde and ring opening of aziridinooxazolidinones.

Despite the variety of methods for preparing amino alcohols, none are suited to all situations. In one aspect, the present invention contemplates a general reduction protocol that benefits from an unappreciated solvent effect. In another aspect, this invention provides a simple preparation of the asymmetric reduction catalyst that requires nothing in the way of complex anaerobic, anhydrous manipulation, purification and/or recrystallization, producing a catalyst that is at once more reactive and more selective than catalyst prepared as described in the literature. In a further aspect, chiral aminoethanols are realized by the agency of intermediate oxazolidinones, which are produced through the reaction of chiral halohydrins with an isocyanate and subsequent cyclization or alternatively might result from the reaction of the chiral halohydrin with a chloroformate, reaction of the derived carbonate with a an amine and subsequent cyclization. The utilization of an intermediate oxazolidinone avoids the production of oligomers and undesired regioisomers, outcomes that are often encountered when a direct amine displacement is attempted. Further, because of the highly polar and often hygroscopic nature of amino alcohols, they are difficult to purify and thus the additional benefits of oxazolidinone formation include simple chiral enrichment by chiral HPLC or recrystallization. The aminoethanols resulting from oxazolidinone cleavage are analytically pure and essentially water free (less than about 99%) as isolated from the reaction. The process consists of the steps 1) the asymmetric reduction of an alpha-halo ketone with a ruthenium complex catalyst in a polar solvent such as dimethylformamide to give a chiral alpha-halohydrin; 2) reacting the alpha-halohydrin of step 1) with an isocyanate (or chloroformate followed by a reaction with an amine) to give the corresponding urethane; 3) contacting the urethane of step 2) with a base to give an oxazolidinone; 4) optionally, purification of the easily manipulated oxazolidinones to provide oxazolidinones of high (>95-99% ee) optical purity; and 5) hydrolysis of the oxazolidinone to provide amino alcohols of high enantiomeric purity.

In one aspect the invention features a method of preparing enantiomerically enriched amino alcohols of Formula I, comprising the steps of:

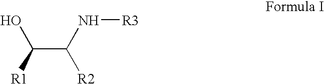

a) reducing a carbonyl compound of Formula A

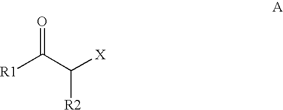

in a solvent in the presence of a reducing agent to give an alcohol of Formula B,

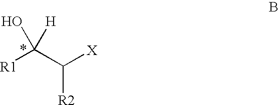

wherein R₁ is alkyl or heteroalkyl of 1-12 carbons, aryl or heteroaryl;

R₂ is H, alkyl of 1-4 carbons, CH₂-Aryl, or CH₂-heteroaryl; and

X is selected from the group Cl, Br, I, Aryl-SO₂O—, perfluoro alkyl-SO₂O— and alkyl-SO₂O—;

b) forming a urethane of Formula D from an alcohol of Formula B

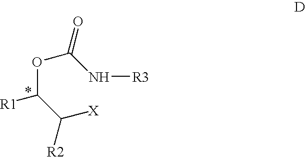

wherein R₃ is selected from the group alkyl of 1-6 carbons, aryl, benzyl, lower alkyl-CO, aryl-CO, lower alkyl-O—CO—, aryl-O—CO—, benzyl-O—CO— and aryl-SO₂—;

c) forming an oxazolidinone of Formula E by treating a urethane of Formula D with a base;

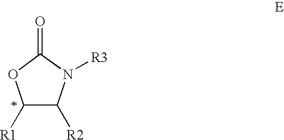

d) purifying an oxazolidinone of Formula E; and

e) converting an oxazolidinone of Formula E to an enantiomerically enriched amino alcohol of Formula 1.

Embodiments of the invention may include one or more of the following features. The reducing agent is a chiral catalyst. The Chiral catalyst includes ruthenium. The chiral catalyst is

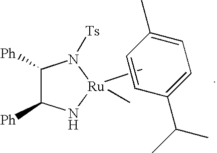

The solvent used in reducing the ketone includes DMF. The urethane of formula D is formed by reacting the alcohol of formula B with an isocyanate of Formula C;

R3NCO C

wherein R₃ is selected from the group alkyl of 1-6 carbons, aryl, benzyl, lower alkyl-CO, aryl-CO, lower alkyl-O—CO—, aryl-O—CO—, benzyl-O—CO— and aryl-SO₂—. The base used to form the oxazolidinone from the urethane of formula D comprises sodium hydride or potassium t-butoxide, sodium amylate, or sodium hydride. The enatiomerically enriched amino alcohol of formula I is greater than about 50% ee, about 80%, about 90% ee, about 95% ee, or about 99% ee.

### DETAILED DESCRIPTION OF THE INVENTION

Definitions

In the detailed description, the following definitions are used. The term “leaving group” means a substituent which is subject to nucleophilic displacement to form a carbon-carbon or heteroatom-carbon bond as described in March, Advanced Organic Chemistry: Reactions, Mechanisms, and Structure, McGraw-Hill, pp. 251-375, 1968. Examples of leaving groups include, but are not limited to, chloro, bromo, iodo, arylsulfonyl and alkylsulfonyl.

The term “ee” means enantiomeric excess. For instance, one enantiomer of a specific compound is present in a mixture of the enantiomers for that compound at a greater amount relative to the other enantiomer. An enantiomerically enriched form may include a mixture of enantiomers of a specific compound in which the concentration of a single enantiomer of that compound is greater than 50%, more typically greater than 60%, 70%, 80%, or 90%, or higher (e.g., >95%, >97%, >99%, >99.5%), relative to the other enantiomer of that compound.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₈ means 1-8 eight carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkene” by itself or as part of another substituent means a divalent radical derived from an alkane, as exemplified by —CH₂CH₂CH₂CH₂—. A “lower alkyl” or “lower alkene” is a shorter chain alkyl or alkene group, having eight or fewer carbon atoms.

The terms “alkoxy . . . alkylacylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “heteroalkyl,” by itself or in combination with another term, means, unless otherwise stated, a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃. Also included in the term “heteroalkyl” are those radicals described in more detail below as “heterocycloalkyl.” The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “halo” or “halogen,” by themselves or as part of another substituent, mean, unless otherwise stated, a fluorine, chlorine, bromine, or iodine atom. Additionally, terms such as “Fluoroalkyl,” are meant to include monofluoroalkyl and polyfluoroalkyl.

The term “aryl,” employed alone or in combination with other terms (e.g., aryloxy, arylthioxy, aralkyl) means, unless otherwise stated, an aromatic substituent, which can be a single ring or multiple rings (up to three rings), which are fused together or linked covalently. The term “heteroaryl” is meant to include those aryl rings which contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include, but are not limited to, phenyl, 1-naphthyl, 2-napthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 2-benzofuranyl, 3-banzofuranyl, 5-benzothiazolyl, purinyl, 2-benzimidazolyl, 1-indolyl, 5-indolyl, 1-isoquinolyl, 5-isoquinolyl, 2-quinoxalinyl, 5-quinoxalinyl, 3-quinolyl, and 6-quinolyl.

Substituents for each of the above noted aryl ring systems are selected from the group of acceptable substituents described below. The term “aralkyl” is meant to include those radicals in which an aryl or heteroaryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl . . . heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, =0, ═NR′, ═N—OR′, —NR′R″—SR′, -halogen,—SiR′R″R, —OC(O)R′, —C(O)R′, —CO₂R′, —CONR′R″, —OC(O)NR′R″—NR′CO)R′, —NR′—C(O)NR″R′″, —NR′COOR″, —NH—C(NH₂)═NH, —NR′C(NH₂)═N—H, —NH—C(NH₂)═NR′, —S(O)R′, S(O)₂R′, —S(O)₂NR′R″, —CN and —NO₂ in a number ranging from zero to (2N+1), where N is the total number of carbon atoms in such radical. R′, R″ and X″ each independently refer to hydrogen, unsubstituted Cl-COalkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 3-7 membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: halogen, —OR, —OC(O)R, —NR′R″, —SR, —R′, —CN, —NO₂, —CO₂R′, —CONR′R:′, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, and (unsubstituted aryloxy-(C₁-C₄)alkyl.

Two of the substituents on adjacent atoms of the aryl ring may optionally be replaced with a substituent of the formula —S—C(O)—(CH₂)_{q}—R—, wherein S and R are independently —NH—, —O—, —CH₂— or a single bond, and the subscript q is an integer of from 0 to 2. Alternatively, two of the substituents on adjacent atoms of the aryl ring may optionally be replaced with a substituent of the formula -A-(CH₂)_{w}—B—, wherein A and B are independently —CH₂—, —O—, —NH—, —S—, —S(O)—, —S(O)₂—, —S(O)₂NR′- or a single bond, and w is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)_{w}-G-(CH₂)_{w}-, where w and w′ are independently integers of from 0 to 3, and G is —O—, —NR′-, —S—, —S(O)—, —S(O)₂—, or —S(O)₂NR′—. The substituent R′ in —NR′- and —S(O)₂NR′— is selected from hydrogen or unsubstituted (C1-C6)alkyl. As used herein, the term “heteroatom” is meant to include oxygen (O), nitrogen (N), and sulfur(S).

### DESCRIPTION OF THE INVENTION

The overall process for producing chiral amino alcohols is summarized in Scheme I.

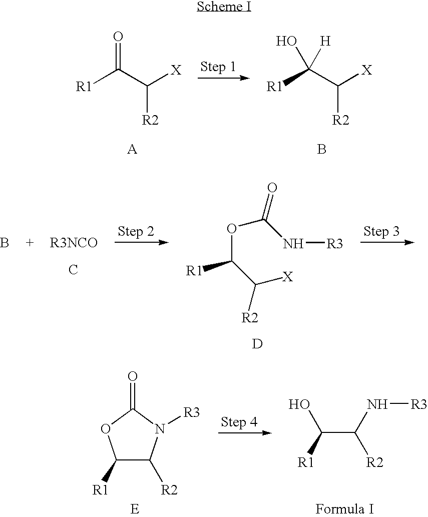

In step 1, a ketone of Formula A,

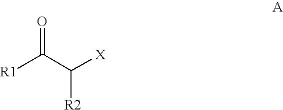

wherein:

R₁ is alkyl or heteroalkyl of 1-12 carbons, aryl or heteroaryl;

R₂ is H, alkyl of 1-4 carbons, CH₂-Aryl, or CH₂-heteroaryl; and

X is selected from the group Cl, Br, I, Aryl-SO₂O—, perfluoro alkyl-SO₂O— and alkyl-SO₂O—; is reduced to a chiral alcohol of Formula B

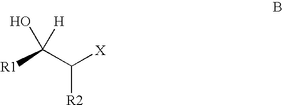

with a suitable chiral reducing reagent.

Methods for achieving the chiral reduction include enantioselective hydride reduction, enantioselective hydrogenation, and enantioselective transfer hydrogenation (see for example Palmer, M. J; et al., Tetrahedron: Asymmetry, (1999), 10, 2045 and references cited therein).

In another aspect of this invention, the ketone A is reduced by enantioselective transfer hydrogenation using a modification of the method described by Noyori, et al. (Noyori, R.; Hashiguchi, S., Accts. Chem. Res., (1997), 30, 97-102; Fujii, A.; Hashiguchi, S.; Uematsu, N.; Ikariya, T.; Noyori, R., J. Am. Chem. Soc. (1996), 118, 2521-2522). The modifications obviate the laborious chiral catalyst preparation and recrystallization as described by Noyori and others (Vedejs, E., et.al., J. Org. Chem. (1999), 64, 6724), and provides a simple, oxygen insensitive, catalyst preparation which enables the preparation of a variety of alcohols of Formula B. The catalyst can be stored or prepared in situ.

The present method also benefits from a heretofore-unappreciated solvent effect. The use of a polar solvent such as dimethylformamide to give elevated yields in shorter time (48 hours reduced to 45 minutes) and with significantly improved enantioselection (ca. 60% ee improved to >99% ee). In preparing the catalyst, a mixture of a suitable ligand such as N-tosyl-1,2-diphenylethylenediamine and a suitable source of ruthenium complex such as RuCl₂(η6-p cymene) dimer in a suitable secondary solvent alcohol such as isopropanol, 2-butanol, cyclohexanol and the like containing a suitable tertiary amine such as triethylamine is heated at 60-80° C. for 1 hour. Evaporation of the solvent gives the desired catalyst as a stable orange-brown solid (Method A). Alternatively, the catalyst can be prepared by combining the ligand, N-tosyl-1,2-diphenylethylenediamine and a ruthenium source such as RuCl₂(η6-p cymene) dimer, in DMF, either DMF only or in the presence of a co-solvent such as methyl-tert-butyl ether (MTBE), followed by the addition of a 5:2 mixture (mole/mole) of formic acid and triethyl amine (Method B). If the reduction is being conducted by the preparation of the catalyst by Method A, the reduction is completed by the addition of polar solvent to the catalyst followed by a ketone of Formula A and a 5:2 to 1:1 (mole/mole) mixture of formic acid and triethylamine and stirring the mixture for 45 minutes to 6 hours, usually 45 minutes, at from −15° C. to room temperature, usually room temperature, at a pressure from 20 mmHg to 1 atm.

In Step 2 of the sequence, the alcohol of Formula B is reacted with an appropriate isocyanate reagent of Formula C;

R3NCO C

wherein R₃ is selected from the group alkyl of 1-6 carbons, aryl, benzyl, lower alkyl-CO, aryl-CO, lower alkyl-O—CO—, aryl-O—CO—, benzyl-O—CO— and aryl-SO₂—; to give the urethane of Formula D

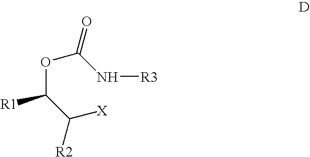

wherein X, R₁, R₂ and R₃ are as defined above. The reaction is optionally conducted in a suitable solvent such as diethyl ether, methylene chloride, chloroform, toluene, dimethoxyethane, tetrahydrofuran and the like at a temperature of from −50° C. to 100° C., usually at 0° C. to 40° C. A tertiary organic base such as triethylamine, pyridine, 4-N,N-dimethylpyridine and the like may be added as a catalyst. Alkyl, aryl, benzyl, acyl, aroyl and arylsulfonyl isocyanates are well known and many are commercially available. Alkoxy, benzyloxy and aryloxy carbonylisocyanates may be prepared by procedures described in U.S. Pat. Nos. 5,386,057 and 4,210,750 the entire contents of which are hereby incorporated by reference.

In Step 3, the urethane of Formula D is reacted with a base such as sodium hydride, potassium t-butoxide and the like in a solvent to give an oxazolidinone of Formula E,

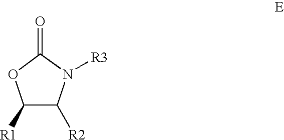

wherein R₁, R₂ and R₃ are as defined above. Suitable bases include, but are not limited to, potassium tert-butoxide, sodium amylate, sodium hydride and the like. Suitable solvents include tert-butyl alcohol, diethyl ether, dimethoxyethane, tetrahydrofuran, dioxane and the like. The reaction is conducted at a temperature of from −50° C. to 100° C., usually at 0° C. to 40° C. The oxazolidinone may be isolated and is readily purified to enhance optical purity by conventional methodology such as recrystallization or chiral high performance liquid chromatography (cf. Cox, G. B. Innov. Pharm. Technol. (2001) 01(8), 131; Issaq, H. J. Prep. Biochem. Biotechnol. (2000), 30(1), 79).

In step 4, the oxazolidinone of Formula E is hydrolyzed to an amino alcohol of Formula I.

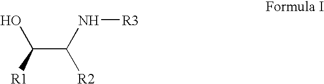

When R₃ is lower alkyl-CO, aryl-CO, lower alkyl-O—CO—, aryl-O—CO—, benzyl-O—CO— and aryl-SO₂— in Formulas D and E, R₃ in Formula I may be lower alkyl-CO, aryl-CO, lower alkyl-O—CO—, aryl-O—CO—, benzyl-O—CO— and aryl-SO₂— or H depending on the particular hydrolysis conditions and substituent.

Hydrolysis is achieved by contacting the oxazolidinone of Formula E with a base such as potassium hydroxide in a protic solvent such as water, ethanol and the like or mixtures of solvents according to standard procedures (Katz, S. J., et.al., Tetrahedron Lett., (2002), 43, 557) When the desired product is an oxazolidinone of Formula I wherein R₃ is lower alkyl-CO, aryl-CO, lower alkyl-O—CO—, aryl-O—CO—, benzyl-O—CO— and aryl-SO₂—, the hydrolysis may be achieved with cesium carbonate in methanol as has been described (Ishizuka, T., et.al., Tetrahedron Lett., (1987), 28, 4185; Benedetti, F., et.al., Tetrahedron Lett., (2000), 41, 10071).

### EXAMPLES

Without further elaboration, it is believed that one skilled in the art can, using the preceding descriptions, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Preparation of R-2-(1-hydroxy-2-chloroethyl)-pyridine

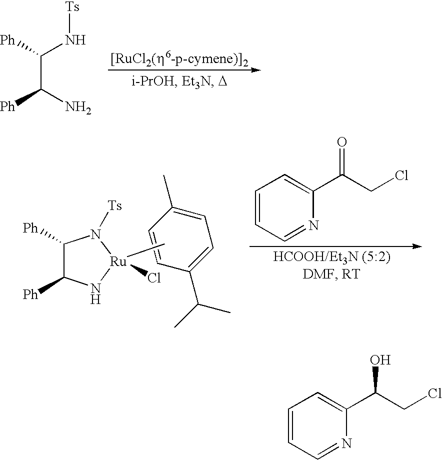

[RuCl₂(η⁶-p-cymene)]₂ (0.84 g, 1.37 mmol), Et₃N (0.67 g, 6.66 mmol, 0.93 mL), and (1S, 2S)—N—P toluenesulfonyl-1,2-diphenylethylenediamine (1.0 g, 2.72 mmol, 1.78 mol % based upon ketone) are combined in a 500 mL 1N round bottom flask. Isopropanol (25 mL) and Et₃N (0.67 g, 6.66 mmol, 0.93 mL) is added, a reflux condenser is attached and the mixture is warmed under reflux, and maintained, for 1 hour. Cool to room temperature and concentrate in vacuo (rotovapor followed by vacuum pump) to furnish the catalyst as a brown powdery solid. To the catalyst is added anhydrous DMF (Aldrich Sure Seal, 225 mL), followed in order by 2-chloroacetylpyridine (23.88 g, 0.153 mol) and HCOOH/Et₃N (5:2, Fluka, 55 mL). After ca. 2-3 minutes of stirring (room temperature) bubbles (presumed to be CO₂) are apparent, emanating from the stirring vortex of the red-black solution. Reaction progress is monitored by reverse phase analytical HPLC, and after 75 minutes of stirring, the starting material had been consumed (95:5 NaH₂PO₄/H₃PO₄ buffered water/CH₃CN to 5:95, 17 minutes; retention time of starting chloroketone: 7.39 minutes, retention time of halohydrin 2.66 minutes). Quench the reaction by adding MeOH (25 mL), stir 5 minutes and then the DMF, etc is removed in vacuo (cold finger rotovapor, vacuum pump) to give a red-black viscous oil. The crude material is taken up in Et₂O/CH₂Cl₂ (4:1, 1.25 L), placed in a 3 L separatory funnel, wash with saturated aq. NaHCO₃ (1.0 L), brine (1.0 L), and dried (Na₂SO₄). Filtration and concentration in vacuo affords the crude product as a red-orange oil which is purified by chromatography on a column of silica gel (70 mm OD, 250 g 230-400 mesh, packed hexanes; compound applied in CH₂Cl₂/hexanes 60:40; eluted with hexanes/Et₂O (75:25 2 L; 65:35 2 L; 55:45 2 L; 350 mL fractions) using the flash technique. Fractions 9-16 are combined to afford 14.72 g (61%) of the target halohydrin as pale yellow solid. Physical Characteristics: MP: 47-48-C; ¹H-NMR (400 MHZ, CDCl₃): δ=8.65, 7.92, 7.58, 7.44, 5.13, 4.60, 3.91; IR (neat): 3138, 3074, 3029, 3014, 2974, 2964, 2955, 2895, 2862, 2848, 2472, 2350, 2328, 2305, 2261 cm⁻¹; Anal. Found: C, 53.23; H, 5.12; N, 8.82; Specific Rotation [α]^{D}₂₅=−39 (c 0.94, CH₂Cl₂); Chiral HPLC Analysis (Chiracel OJ): 98:2; 96% ee.

### Example 2

### S-2-(1-hydroxy-2-chloroethyl)-pyridine

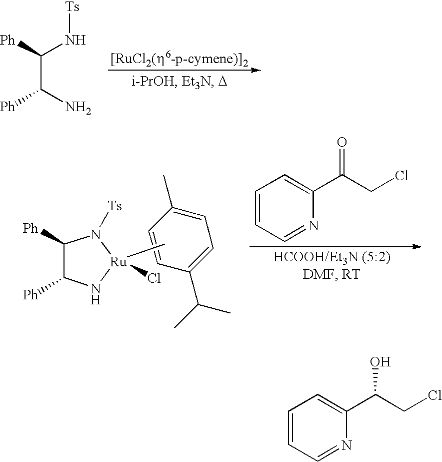

[RUCl₂(η⁶-p-cymene)]₂ (0.84 g, 1.37 mmol), Et₃N (0.679, 6.66 mmol, 0.93 mL), and (1 R, 2 R)-N-p-toluenesulfonyl-1,2-diphenylethylenediamine (1.0 g, 2.72 mmol, 1.78 mol % based upon ketone) are combined in a 500 mL 1N round bottom flask. i—PrOH (25 mL) and Et₃N (0.67 g, 6.66 mmol, 0.93 mL) are added, a reflux condenser is attached and the mixture is warmed under reflux, and maintained, for 1 hour. Cool to room temperature and concentrate in vacuo (rotovapor followed by vacuum pump) to furnish the catalyst as a brown powdery solid. To the catalyst is added anhydrous DMF (Aldrich Sure Seal, 225 mL), followed in order by 2-chloroacetylpyridine (23.88 g, 0.153 mol) and HCOOH/Et₃N (5:2, Fluka, 55 mL). After ca. 2-3 minutes of stirring (room temperature) bubbles (presumed to be CO₂) are apparent, emanating from the stirring vortex of the red-black solution. Reaction progress is monitored by reverse phase analytical HPLC, and after 65 minutes of stirring, the starting material had been consumed (95:5 NaH₂PO₄/H₃PO₄ buffered water/CH₃CN to 5:95, 17 minutes; retention time of starting chloroketone: 7.39 minutes, retention time of halohydrin 2.66 minutes). Quench the reaction by adding MeOH (25 mL), stir 5 minutes and then the DMF, etc is removed in vacuo (cold finger rotovapor, vacuum pump) to give a red-black viscous oil. The crude material is taken up in Et₂O/CH₂Cl₂ (4:1, 1.25 L), placed in a 3 L separatory funnel, wash with saturated aq. NaHCO₃ (1.0 L), brine (1.0 L), and dried (Na₂SO₄). Filtration and concentration in vacuo affords the crude product as a red-orange oil which is purified by chromatography on a column of silica gel (70 mm OD, 250 g 230-400 mesh, packed hexanes; compound applied in CH₂Cl₂/hexanes 60:40; eluted with hexanes/Et₂O (75:25 2 L; 65:35 2 L; 55:45 2 L; 350 mL fractions) using the flash technique. Fractions 11-17 are combined to afford 16.419 (68%) of the target halohydrin as pale yellow solid. Physical Characteristics: MP: 49-50° C.; ¹H-NMR (400 MHz, CDCl₃): δ=8.60, 7.77, 7.58, 7.30, 5.00, 4.20, 3.85; EI-MS (70 EV): 160(35), 158(M⁺, 90), 122(90), 106(base); IR (neat): 3085, 3075, 2470, 2350, 2328, 2305, 2260, 1109, 1077, 1006, 783, 762, 720, 640, 624 cm⁻¹; Anal. Found: C, 53.27; H, 5.19; N, 8.81, Cl, 22.29; Specific Rotation [α]D₂₅=62 (c 0.94, methanol); Chiral HPLC Analysis (Chiracel OJ): 100:0; >99% ee.

### Example 3

### S-2-(1-hydroxy-2-N-methylamino-ethyl)-pyridine

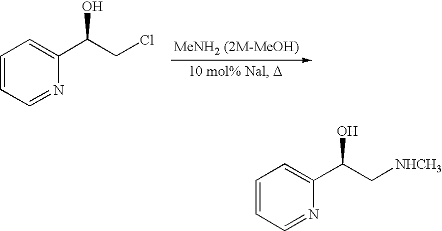

R-2-(1-hydroxy-2-chloroethyl)-pyridine (6.0 g, 38 mmol) and NaI (0.57 g, 3.8 mmol) are combined in a 500 mL, plastic coated, thick walled bottle and are covered with 2M MeNH₂ in MeOH (0.19 L). The Teflon stopper is wrapped in Teflon tape, the bottle is sealed. Stirring is started, and the bottle is immersed in a 60° C. oil bath for 16 hours. The yellow-brown mixture is cooled to room temperature and analyzed by analytical reverse phase HPLC, which indicated that the reaction is complete (retention time starting material=2.66 minutes; retention time product=1.22 minutes). Concentration in vacuo affords the crude product as a yellow oil, which is treated with CH₂Cl₂-THF (0.25 L, 10:90) to give a yellow solution and a whit precipitate. The precipitate is removed by filtration, is rinsed with CH₂Cl₂-THF (10:90) and the combined filtrated are concentrated in vacuo to give a yellow-brown oil. The crude product is purified by chromatography on a column of silica gel (70 mm OD, 250 g, 230-400 mesh; packed with CH₂Cl₂-MeOH 90:10; eluted with CH₂Cl₂-MeOH 90:10, 2 L, 500 mL fractions; CH₂Cl₂-MeOH—NH₄OH 89:10:1, 8 L, 500 mL fractions) using the flash technique. Fractions 10-18 are combined to provide 3.34 g (58%) of the target aminoethanol as an amber oil. Physical Characteristics: ¹H-NMR (400 MHz, DMSO-d₆): δ=8.48, 7.78, 7.50, 7.30, 4.70, 2.85, 2.67, 2.34; EI-MS (70 EV): 153(base), 135(20), 122(27), 108(43); IR (neat): 3291, 3090, 3066, 2942, 2890, 2853, 2799, 1996, 1918, 1591, 1473, 1436, 1070, 772, 751 cm⁻¹; HRMS (FAB): found 153.1046; Specific Rotation [α^{D}₂₅]=46 (c 0.37, CH₂Cl₂).

### Example 4

### R-2-(1-hydroxy-2-N-methylamino-ethyl)-pyridine

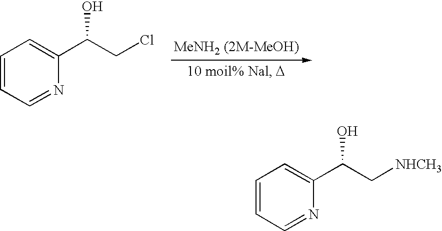

S-2-(1-hydroxy-2-chloroethyl)-pyridine (6.0 g, 38 mmol) and NaI (0.57 g, 3.8 mmol) are combined in a 500 mL, plastic coated, thick walled bottle and are covered with 2M MeNH₂ in MeOH (0.19 L). The Teflon stopper is wrapped in Teflon tape, the bottle is sealed. Stirring is started, and the bottle is immersed in a 60° C. oil bath for 16 hours. The yellow-brown mixture is cooled to room temperature and analyzed by analytical reverse phase HPLC, which indicated that the reaction is complete (retention time starting material=2.44 minutes; retention time product=1.24 minutes). Concentration in vacuo affords the crude product as a yellow oil, which is treated with CH₂Cl₂-THF (0.25 L, 10:90) to give a yellow solution and a white precipitate. The precipitate is removed by filtration, is rinsed with CH₂Cl₂-THF (10:90) and the combined filtrated are concentrated in vacuo to give a yellow-brown oil. The crude product is purified by chromatography on a column of silica gel (70 mm OD, 250 g, 230-400 mesh; packed with CH₂Cl₂-MeOH 90:10; eluted with CH₂Cl₂-MeOH 90:10, 2 L, 500 mL fractions; CH₂Cl₂-MeOH—NH₄OH 89:10:1, 8 L, 350 mL fractions) using the flash technique. Fractions 14-30 are combined to provide 3.189 (54%) of the target aminoethanol as an amber oil. Physical Characteristics: ¹H-NMR (400 MHz, DMSO-d₆): δ=8.49, 7.79, 7.52, 7.25, 4.75, 2.90, 2.67, 2.32; EI-MS (70 EV): 153(base), 135(18), 122(20), 108(62); IR (neat): 3279, 3090, 3064, 3012, 2943, 2890, 2851, 2799, 1996, 1591, 1473, 1436, 1070, 772, 751 cm⁻¹;

HRMS (FAB): found 153.1009; Specific Rotation [α^{D}₂₅]=49 (c 0.36, CH₂Cl₂).

### Example 5

### 2-[1-Tri-isopropylsilyloxy-vinyl]-furan

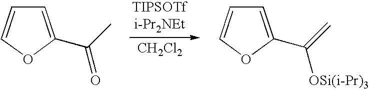

2-Acetylfuran(50 g (0.454 mol) is placed in a 2 L 1N round bottom flask and anhydrous CH₂Cl₂ (Aldrich Sure Seal, 0.70 L) is added, followed by the addition of i-Pr₂NEt (176 g, 1.36 mol, 3 eq., 237 mL). The flask is equipped with a 125 mL pressure equalized dropping funnel, and the mixture is placed under nitrogen and cooled in an ice-water bath. To the chilled ketone/amine mixture is added TIPSOTf (153.2 g, 0.5 mol, 1.1 eq., 134.3 mL) over 1.5 hours. The mixture is allowed to warm to room temperature overnight. The reaction mixture is concentrated in vacuo on a rotary evaporator (T≦25° C.) to give a yellow oil and a white solid. The flask contents are transferred to a 2 L separatory funnel with ether (1.2 L) resulting in the formation of additional white solid material (likely iPr₂(Et)NH⁺⁻OTf which might be removed by filtration but is not in this experiment) and the mixture is wash with saturated aq. NaHCO₃ (2×0.70 L). The organic phase is separated, dried over Na₂SO₄, then is concentrated in vacuo to furnish the crude enol ether (1 18.3 g, 98%) as a yellow-orange oil. This crude material is not further purified, but is immediately carried to the next step. Physical Characteristics: ¹H-NMR (400 MHz, CDCl₃): δ=7.36, 6.49, 6.40, 4.86, 4.37, 1.32, 1.14.

### Example 6

### 2-[1-Tri-isopropylsilyloxy-2-chloro-vinyl]-furan

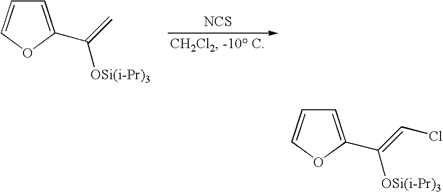

2-[1-Tri-isopropylsilyloxy-vinyl]-furan (116.3 g, assumed 0.436 mmol) is placed in a 2 L, IN round bottom flask and dissolved in anhydrous THF (Aldrich Sure Seal, 0.6 L). The flask is placed under nitrogen, cooled in a −10° C. bath, then NCS (64.11 g, 0.48 mol, 1.1 eq.) is added and the mixture is stirred for 1 hour, after which time the reaction is judged to be complete by analytical reverse phase HPLC. The reaction mixture is warmed to room temperature, poured into a 4 L separatory funnel containing ether (1.5 L), and is wash with saturated aq. NaHCO₃ (2×0.7 L). The organic phase is separated, dried (Na₂SO₄), and concentrated in vacuo to afford the target chloro-enol ether (129.9 g, 99%) as a yellow-orange oil. The crude material is not further purified, but is immediately carried into the next step. Physical Characteristics: ¹H-NMR (400 MHz, CDCl₃): δ=7.36, 6.43, 6.40, 5.95, 1.30, 1.1 1.

### Example 7

### 2-Chloroacetylfuran

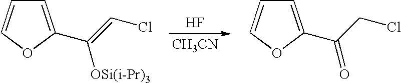

2-[1-Tri-isopropylsilyloxy-2-chloro-vinyl]-furan (129.9 g, 0.431 mol) is placed in a 4 L plastic bottle and is dissolved in acetonitrile (0.6 L). To the stirring solution is added 48% aqueous HF (65 mL, 0.15 mL/mmol) and the progress of the reaction is monitored by reverse phase analytical HPLC. After. Ca. 2 hours the reaction is judged to be complete, and the pH of the solution is carefully adjusted to ca. 7 with saturated aq. NaHCO₃. The mixture is poured into a separatory funnel containing CH₂Cl₂ (1.5 L). The organic phase is removed and the aq. layer is extracted with CH₂Cl₂ (2×1.0 L). The combined organic layers are dried (Na₂SO₄), and concentration in vacuo affords the crude 2-chloroacetyl furan (41.9 g, 67%) as a yellow oil. The crude material is judged to be quite pure by ¹H-NMR and HPLC and is used as is in the Noyori asymmetric reduction. Physical Characteristics: ¹H-NMR (400 MHz, CDCl₃): δ=7.58, 7.33, 6.59, 4.57; MS (ES+): 145.4.

### Example 8

### S-1-(2-furyl)-2-chloroethanol

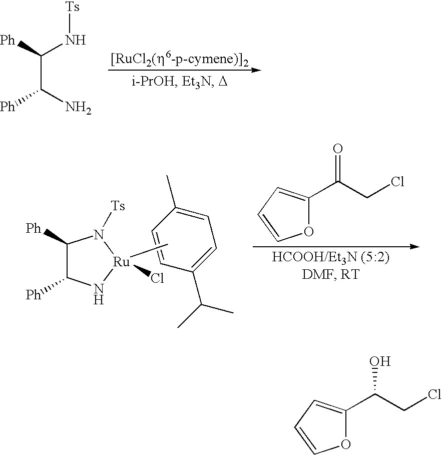

[RuCl₂(η⁶-p-cymene)]₂ (0.999, 1.61 mmol), Et₃N (0.67 g, 6.66 mmol, 0.93 mL), and (1R, 2R)-N-p-toluenesulfonyl-1,2-diphenylethylenediamine (1.18 g, 3.22 mmol, 2.25 mol % based upon ketone) are combined in a 500 mL 1N round bottom flask. i-PrOH (25 mL) and Et₃N (0.67 g, 6.66 mmol, 0.93 mL) are added, a reflux condenser is attached and the mixture is warmed under reflux, and maintained, for 1 hour. Cool to room temperature and concentrate in vacuo (rotovapor) to furnish the catalyst as an orange-brown powdery solid. To the catalyst is added anhydrous DMF (Aldrich Sure Seal, 250 mL), followed in order by 2-chloroacetylfuran (20.6 g, 0.143 mol) and HCOOH/Et₃N (5:2, Fluka, 51 mL). After ca. 2-3 minutes of stirring (room temperature) bubbles (presumed to be CO₂) are apparent, emanating from the stirring vortex of the red-black solution. Reaction progress is monitored by reverse phase analytical HPLC, and after 65 minutes of stirring, the starting material had been consumed (95:5 NaH₂PO₄/H₃PO₄ buffered water/CH₃CN to 5:95, 17 minutes; retention time of starting chloroketone: 6.70 minutes, retention time of halohydrin 6.35 minutes). Quench the reaction by adding MeOH (25 mL), stir 5 minutes and then the reaction mixture is poured into ice-water (1 L) and the aqueous phase is saturated with salt. The mixture is transferred to a 2 L separatory funnel with ether (500 mL), shaken, and the organic phase is removed. The aqueous layer is extracted with ether (3×250 mL) and the combined organic layers are wash with saturated aq. NaHCO₃ (0.5 L), brine (4×250 mL), and dried (Na₂SO₄). Filtration and concentration in vacuo affords the crude product as a red-orange oil (20.5 g) that is triturated with ether/pentane (10:90, 4×100 mL). The combined triturates are concentrated in vacuo (take care as the halohydrin is volatile, hence the choice of ether/pentane as triturant and no removal of DMF in vacuo) to furnish the desired halohydrin S-1-(2-furyl)-2-chloroethanol (15.97 g, 76%) in good purity as determined by HPLC and ¹H-NMR. Physical Characteristics:

¹H-NMR (400 MHz, CDCl₃): δ=7.41, 6.37, 4.95, 3.85, 2.58; IR (diffuse reflectance) 1428, 1422, 1221, 1205, 1198, 1166, 1096, 1021, 953, 924, 883, 789, 738, 714, 666, cm⁻¹; MS (EI) m/z (rel. intensity) 146 (17), 129 (2), 98 (6), 97 (base), 95 (3), 94 (1), 69 (3), 41 (2); HRMS (EI) found 146.0136; Specific Rotation [α^{D}₂₅]=17 (c 0.97, methanol); Chiral HPLC Analysis (Chiracel OJ): 99:1; 98% ee.

### Example 9

### R-1-(2-furyl)-2-chloroethanol

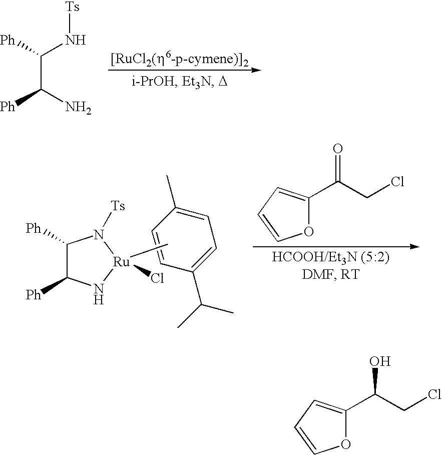

[RuCl₂(η⁶-p-cymene)]₂ (0.999, 1.61 mmol), Et₃N (0.67 g, 6.66 mmol, 0.93 mL), and (1 S, 2S)—N—P toluenesulfonyl-1,2-diphenylethylenediamine (1.18 g, 3.22 mmol, 2.10 mol % based upon ketone) are combined in a 500 mL 1N round bottom flask. i-PrOH (25 mL) and Et₃N (0.67 g, 6.66 mmol, 0.93 mL) are added, a reflux condenser is attached and the mixture is warmed under reflux, and maintained, for 1 hour. Cool to room temperature and concentrate in vacuo (rotovapor) to furnish the catalyst as an orange-brown powdery solid. To the catalyst is added anhydrous DMF (Aldrich Sure Seal®, 250 mL), followed in order by 2-chloroacetylfuran (22.3 g, 0.154 mol) and HCOOH/Et₃N (5:2, Fluka, 55 mL). After ca. 2-3 minutes of stirring (room temperature) bubbles (presumed to be CO₂) are apparent, emanating from the stirring vortex of the red-black solution. Reaction progress is monitored by reverse phase analytical HPLC, and after 65 minutes of stirring, the starting material had been consumed (95:5 NaH₂PO₄/H₃PO₄ buffered water/CH₃CN to 5:95, 17 minutes; retention time of starting chloroketone: 6.70 minutes, retention time of halohydrin 6.35 minutes). Quench the reaction by adding MeOH (25 mL), stir 5 minutes and then the reaction mixture is poured into ice-water (1 L) and the aqueous phase is saturated with salt. The mixture is transferred to a 2 L separatory funnel with ether (500 mL), shaken, and the organic phase is removed. The aqueous layer is extracted with ether (3×250 mL) and the combined organic layers are wash with saturated aq. NaHCO₃ (0.5 L), brine (4×250 mL), and dried (Na₂SO₄). Filtration and concentration in vacuo affords the crude product as a red-orange oil (22.7 g) that is triturated with ether/pentane (10:90, 4×100 mL). The combined triturates are concentrated in vacuo (take care as the halohydrin is volatile, hence the choice of ether/pentane as triturant and no removal of DMF in vacuo) to furnish the desired halohydrin R-1-(2-furyl)-2-chloroethanol (16.03 g, 71%) in good purity as determined by HPLC and ¹H-NMR. Physical Characteristics: ¹H-NMR (400 MHz, CDCl₃): δ=7.41, 6.32, 4.92, 3.82, 2.58; IR (liq.) 3373, 2475, 2084, 2023, 1940, 1505, 1226, 1151, 1142, 1089, 1068, 1012, 884, 818, 742 cm⁻¹; MS (EI) m/z (rel. intensity) 146 (13), 148 (4), 146 (13), 98 (4), 97 (base), 95 (4), 94 (2), 69 (6), 65 (2), 41 (7), 39 (3); HRMS (EI) found 146.0133; Specific Rotation [UD ₂₅]=−18 (c 0.97, methanol); Chiral HPLC Analysis (Chiracel OJ): 99:1; 98% ee.

### Example 10

### S-1-(2-furyl)-2-chloroethanol-N-methylcarbamate

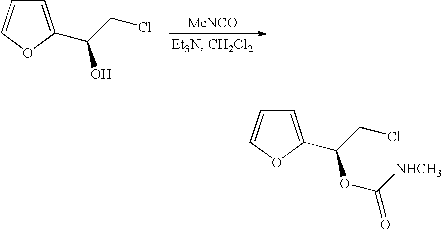

To— 1-(2-furyl)-2-chloroethanol (5.0 g, 34.2 mmol) in dry CH₂Cl₂ (Aldrich Sure Seals, 75 mL), cooled in an ice-water bath under nitrogen, is added Et₃N (1.38 g, 13.7 mmol, 0.4eq., 1.9 mL). Stir 5 minutes, then methylisocyanate (3.32 g, 58.21 mmol, 1.7eq., 3.46 mL) is added via syringe over 2 minutes. Allow the ice to melt and the mixture top warm toward room temperature while monitoring the reaction by HPLC. At 45 minutes the reaction is ca. 35% complete (halohydrin retention time=6.355 min.; product RT=7.826 min.). Allow to stir overnight, HPLC at 16 hours indicated that the reaction is complete. The mixture is cast into Et₂O (0.3 L) and brine (0.3 L). The organic phase is reserved, the aq. Layer is extracted with Et₂O (2×0.2 L), the combined organic phases are wash with brine (0.4 L), and dried (Na₂SO₄). Concentration in vacuo affords the crude carbamate as a brown viscous oil which is purified by chromatography (Biotage® 40 g column, EtOAc/hexanes 10:90 1 L, EtOAc/hexanes 20:80 1 L, 50 mL fractions). Fractions 25-42 affords 4.56 g (65%) of S-1-(2-furyl)-2-chloroethanol-N-methylcarbamate as a clear, pale yellow oil which solidified to an ivory solid upon cooling. Physical Characteristics: MP: 26-27° C.; ¹H-NMR (400 MHz, CDCl₃):δ=7.43, 6.45, 6.39, 5.97, 4.79, 3.89, 2.82; ¹³C-NMR (100 MHz, CDCl₃): δ=156.2, 150.3, 143.3, 110.8, 109.9, 69.1, 44.0, 28.0; IR (diffuse reflectance): 3365, 3355, 3344, 3333, 2477, 2392, 2197, 2088, 1727, 1694, 1550, 1531, 1518, 1253, 1248 cm⁻¹; MS (CI) m/z (rel. intensity): 221 (3), 146 (7), 129 (6), 113 (5), 96 (base), 79 (53), 52 (33); Anal. Found: C, 46.99; H, 4.89; N, 6.85; Cl, 17.31; Specific Rotation [α^{D}₂₅]94 (c 1.02, CH₂Cl₂); Chiral HPLC Analysis (Chiracel OJ): 99:1; 98% ee.

### Example 11

### R-1-(2-furyl)-2-chloroethanol-N-methylcarbamate

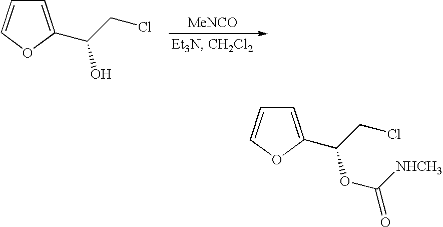

To (R)-1-(2-furyl)-2-chloroethanol (5.0 g, 34.2 mmol) in dry CH₂Cl₂ (Aldrich Sure Seal®, 75 mL), cooled in an ice-water bath under nitrogen, is added Et₃N (1.38 g, 13.7 mmol, 0.4 eq., 1.9 mL). Stir 5 minutes, then methylisocyanate (3.32 g, 58.21 mmol, 1.7 eq., 3.46 mL) is added via syringe over 2 minutes. Allow the ice to melt and the mixture top warm toward room temperature while monitoring the reaction by HPLC. At 45 minutes the reaction is ca. 35% complete (halohydrin retention time=6.355 min.; product RT=7.826 min.). Allow to stir overnight, HPLC at 16 hours indicated that the reaction is complete. The mixture is cast into Et₂O (0.3 L) and brine (0.3 L). The organic phase is reserved, the aq. Layer is extracted with Et₂O (2×0.2 L), the combined organic phases are wash with brine (0.4 L), and dried (Na₂SO₄). Concentration in vacuo affords the crude carbamate as a brown viscous oil which is purified by chromatography (Biotage®D 40 g column, EtOAc/hexanes 10:90 1 L, EtOAc/hexanes 20:80 1 L, 50 mL fractions). Fractions 25-42 affords 5.06 g (73%) of R-1-(2-furyl)-2-chloroethanol-N-methylcarbamate as a clear, pale yellow oil which solidified to an ivory solid upon cooling. Physical Characteristics: MP: 26-27° C.; ¹H-NMR (400 MHz, CDCl₃): δ=7.41, 6.43, 6.40, 5.96, 4.91, 3.87, 2.81; ¹³C-NMR (100 MHz, CDCl₃): δ=156.2, 150.3, 143.3, 110.8, 109.9, 69.1, 44.0, 28.0; IR (diffuse reflectance): 3365, 3355, 3344, 3333, 2477, 2392, 2197, 2088, 1727, 1694, 1550, 1531, 1518, 1253, 1248, cm⁻¹; MS (CI) m/z (rel. intensity): 221 (50), 146 (26), 129 (28), 110 (20), 95 (34), 52 (base); Anal. Found: C, 46.97; H, 4.95; N, 6.90; Cl, 17.27. Specific Rotation [α^{D}₂₅]=−99 (c 0.93, CH₂Cl₂); Chiral HPLC Analysis (Chiracel OJ): 1:99; 98% ee.

### Example 12

### 5R-3-Methyl-5-(2-furyl)-2-oxazoldinone

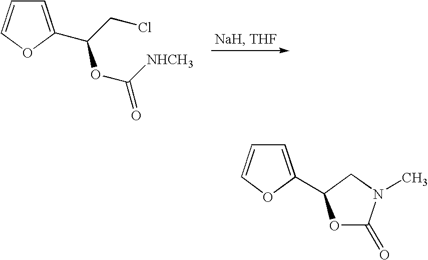

Sodium hydride (1.18 g, 60% in oil, 29.54 mmol) is added to a dried 100 mL, 1 neck 14/20 round bottom flask, equipped with a 50 mL pressure equalized addition funnel, the NaH is covered with dry THF (15 mL, Aldrich Sure Seal®), and the apparatus is placed under nitrogen. The addition funnel is charged with S-1-(2-furyl)-2-chloroethanol-N-methylcarbamate (3.00 g, 14.77 mmol) dissolved in dry THF (25 mL) and the flask is cooled in an ice-water bath. The contents of the addition funnel are then added over 0.5 hour and the mixture is allowed to stir (ice-water cooling) while the reaction is monitored by HPLC. At the end of 1 hour the reaction is judged to be complete (carbamate RT=7.826 min; product RT=5.836 min.), the reaction is carefully quenched by adding 1N aq. HCl (15 mL) and the mixture is poured into CH₂Cl₂ (0.4 L) and brine (0.5 L). The organic phase is separated, dried (Na₂SO₄), and concentrated in vacuo to give the crude oxazolidinone as a yellow oil, overlain by the oil from the NaH. The crude material is purified by chromatography on a 90 g Biotage® column (CH2C12, 1 L; Et₂O:CH₂Cl₂ 2:98, 1 L; Et₂O:CH₂Cl₂ 4:96, 1 L; Et₂O:CH₂Cl₂ 6:94, 1 L; 50 mL fractions). Fractions 23-57 are combined to afford 2.29 g (93%) of 5 R-3-Methyl-5-(2-furyl)-2-oxazoldinone as a pale yellow oil, which solidified to furnish an ivory solid upon cooling. Physical Characteristics: MP: 54-55° C.; ¹H-NMR (400 MHz, CDCl₃): δ=7.47, 6.49, 6.41, 5.46, 3.78, 2.97; ¹³C-NMR (100 MHz, CDCl₃):δ=155.9, 148.1, 142.1, 109.0, 108.4, 65.9, 48.8, 29.4; IR (diffuse reflectance): 2492, 2436, 2402, 2351, 2304, 1759, 1743, 1503, 1439, 1307, 1267, 1154, 1138, 1029,747, cm⁻¹; MS (EI) m/z (rel. intensity): 167 (71), 167 (71), 123 (base), 108 (76), 95 (43), 94 (59), 86 (45), 84 (64), 81 (70), 53 (28), 51 (50); Anal. Found: C, 57.46; H, 5.39; N, 8.36; Specific Rotation [α^{D}₂₅]=−106 (c 1.01, CH₂Cl₂); Chiral HPLC Analysis (Chiracel OJ): 2.8:97.2; 94.4% ee.

### Example 13

### 5S-3-Methyl-5-(2-furyl)-2-oxazoldinone

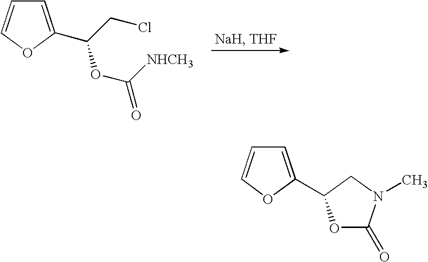

Sodium hydride (1.18 g, 60% in oil, 29.54 mmol) is added to a dried 100 mL, 1 neck 14/20 round bottom flask, equipped with a 50 mL pressure equalized addition funnel, the NaH is covered with dry THF (15 mL, Aldrich Sure Seal®), and the apparatus is placed under nitrogen. The addition funnel is charged with R-1-(2-furyl)-2-chloroethanol-N-methylcarbamate (3.00 g, 14.77 mmol) dissolved in dry THF (25 mL) and the flask is cooled in an ice-water bath. The contents of the addition funnel are then added over 0.5 hour and the mixture is allowed to stir (ice-water cooling) while the reaction is monitored by HPLC. At the end of 1 hour the reaction is judged to be complete (carbamate RT=7.826 min; product RT=5.836 min.), the reaction is carefully quenched by adding 1N aq. HCl (15 mL) and the mixture is poured into CH₂Cl₂ (0.4 L) and brine (0.5 L). The organic phase is separated, dried (Na₂SO₄), and concentrated in vacuo to give the crude oxazolidinone as a yellow oil, overlain by the oil from the NaH. The crude material is purified by chromatography on a 90 g Biotage® column (CH2Cl2, 1 L; Et₂O:CH₂Cl₂ 2:98, 1 L; Et₂O:CH₂Cl₂ 4:96, 1 L; Et₂O:CH₂Cl₂ 6:94, 1 L; 50 mL fractions). Fractions 23-57 are combined to afford 2.29 g (93%) of 5 S-3-Methyl-5-(2-furyl)-2-oxazoldinone as a pale yellow oil, which solidified to furnish an ivory solid upon cooling. Physical Characteristics: MP: 54-55° C.; ¹H-NMR (400 MHz, CDCl₃): δ=7.47, 6.50, 6.41, 5.48, 3.79, 2.97; ¹³C-NMR (100 MHz, CDCl₃):δ=155.9, 148.1, 142.1, 109.0, 108.4, 65.9, 48.8, 29.4; IR (diffuse reflectance): 2491, 2464, 2436, 2402, 2351, 1743, 1503, 1439, 1344, 1307, 1267, 1154, 1138, 1029, 748, cm⁻¹; MS (EI) m/z (rel. intensity): 167 (57), 167 (57), 123 (69), 108 (44), 95 (26), 94 (37), 86 (67), 84 (base), 81 (43), 53 (20), 51 (57); Anal. Found: C, 57.42; H, 5.48; N, 8.38; Specific Rotation [α^{D}₂₅]=109 (c 0.97, CH₂Cl₂); Chiral HPLC Analysis (Chiracel OJ): 98.5:1.5; 97% ee.

Alternatively, the oxazolidinones cited above could be prepared without carbamate purification, utilizing KOtBu as the base as follows:

### Example 14

### 5 R-3-Methyl-5-(2-furyl)-2-oxazoldinone

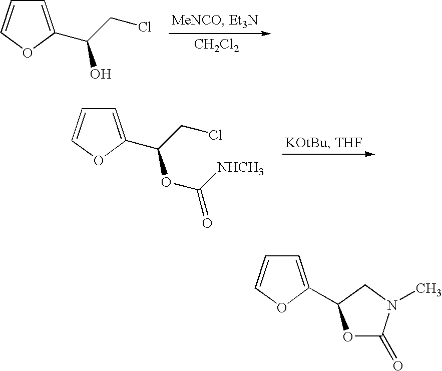

To (S)-1-(2-furyl)-2-chloroethanol (14.0 g, 95.88 mmol) in dry CH₂Cl₂ (Aldrich Sure Seal®, 200 mL), cooled in an ice-water bath under nitrogen, is added Et₃N (3.88 g, 38.3 mmol, 0.4 eq., 5.34 mL). Stir 5 minutes, then methylisocyanate (9.3 g, 163 mmol, 1.7eq., 9.7 mL) is added via. syringe over 5 minutes. Allow the ice to melt and the mixture top warm toward room temperature while monitoring the reaction by HPLC. At 45 minutes the reaction is ca. 35% complete (halohydrin retention time=6.355 min.; product RT=7.826 min.). Allow to stir for an additional 3.25 hours at which point, HPLC indicated that the reaction is complete. The mixture is cast into Et₂O (1.0 L) and brine (1.0 L). The organic phase is reserved, the aq. Layer is extracted with Et₂O (2×0.5 L), the combined organic phases are wash with brine (1.5 L), and dried (Na₂SO₄). Concentration in vacuo affords the crude carbamate as a brown viscous oil which is purified utilized in the cyclization without further purification.

The crude carbamate, from 95.88 mmol of—1-(2-furyl)-2-chloroethanol is dissolved in dry THF (0.2 L, Aldrich Sure Seal®) and the solution is cooled in an ice-water bath under nitrogen. To the chilled carbamate solution is added KOtBu (1.0M in THF, 97 mL, 97 mmol, 1.01 eq.) over 15 minutes. The mixture is allowed to stir after the addition is complete and HPLC analysis suggested that the reaction is complete within 15 minutes. The mixture is cast into Et₂O (1.25 L) and brine (1.0 L) containing 1N aq. HCL (50 mL). The organic phase is separated, the aqueous layer is extracted with Et₂O (1.0 L). The combined organic phases are wash with saturated aq. NaHCO₃ (1.0 L) and dried (Na₂SO₄). Concentration in vacuo affords the crude oxazolidinone as a red-black oil which is triturated with pentane-Et₂O (2:1; 3×0.2 L). The pentane-Et₂O aliquots are concentrated in vacuo to give a red solid which is purified by chromatography on a 120 g Biotage® column (introduced as a solution in CH₂Cl₂, eluted with EtOAc/hexanes, 35:65, 1.0 L; EtOAc/hexanes, 50:50, 2.0 L, 50 mL fractions). Fractions 21-45 are combined to afford 8.75 g (55% from halohydrin) of 5 R-3-Methyl-5-(2-furyl)-2-oxazoldinone as an ivory solid.

### Example 15

### 5 S-3-Methyl-5-(2-furyl)-2-oxazoldinone

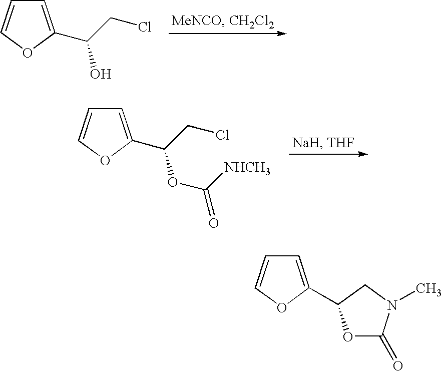

To (R)-1-(2-furyl)-2-chloroethanol (10.09 g, 69.1 mmol) in dry CH₂Cl₂ (Aldrich Sure Seal®, 150 mL), cooled in an ice-water bath under nitrogen, is added Et₃N (2.80 g, 27.6 mmol, 0.4 eq., 3.85 mL). Stir 5 minutes, then methylisocyanate (6.7 g, 117 mmol, 1.7 eq., 7.0 mL) is added via syringe over 5 minutes. Allow the ice to melt and the mixture top warm toward room temperature while monitoring the reaction by HPLC. At 45 minutes the reaction is ca. 35% complete (halohydrin retention time=6.355 min.; product RT=7.826 min.). Allow to stir for an additional 3.25 hours at which point, HPLC indicated that the reaction is complete. The mixture is cast into Et₂O (1.0 L) and brine (1.0 L). The organic phase is reserved, the aq. Layer is extracted with Et₂O (2×0.5 L), the combined organic phases are wash with brine (1.5 L), and dried (Na₂SO₄). Concentration in vacuo affords the crude carbamate as a brown viscous oil which is purified utilized in the cyclization without further purification. The crude carbamate, from 69.1 mmol of (R)-1-(2-furyl)-2-chloroethanol is dissolved in dry THF (0.15 L, Aldrich Sure Seal®)) and the solution is cooled in an ice-water bath under nitrogen. To the chilled carbamate solution is added KOtBu (1.0M in THF, 70 mL, 70 mmol, 1.01 eq.) over 15 minutes. The mixture is allowed to stir after the addition is complete and HPLC analysis suggested that the reaction is complete within 15 minutes. The mixture is cast into Et₂O (1.25 L) and brine (1.0 L) containing 1N aq. HCL (50 mL). The organic phase is separated, the aqueous layer is extracted with Et₂O (1.0 L). The combined organic phases are wash with saturated aq. NaHCO₃ (1.0 L) and dried (Na₂SO₄)—Concentration in vacuo affords the crude oxazolidinone as a red-black oil which is triturated with pentane-Et₂O (2:1; 3×0.2 L). The pentane-Et₂O aliquots are concentrated in vacuo to give a red solid which is purified by chromatography on a 120 g Biotage® column (introduced as a solution in CH₂Cl₂, eluted with EtOAc/hexanes, 35:65, 1.0 L; EtOAc/hexanes, 50:50, 2.0 L, 50 mL fractions). Fractions 23-39 are combined to afford 7.42 g (64% from halohydrin) of 5S-3-Methyl-5-(2-furyl)-2-oxazoldinone as an ivory solid.

### Example 16

### N-Methyl R-1-(2-furyl)-2-aminoethanol

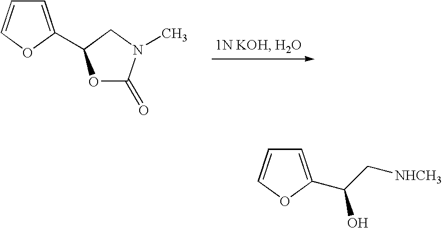

To 5R-3-Methyl-5-(2-furyl)-2-oxazoldinone (8.09, 47.8 mmol) in a 500 mL 1 N RB flask is added 1N aq. KOH (240 mL, 0.24 mol, 5 eq.). The flask is equipped with a reflux condenser, placed under nitrogen, then is immersed in a preheated (50° C.) oil bath. The mixture is allowed to stir and the PHA-727185 suspension slowly gave way to a clear solution. After stirring for 3 hours at 50° C. HPLC analysis indicated that the reaction is complete. The mixture is cooled to room temperature and is cast into a separatory funnel, the flask is rinsed into the separatory funnel with Et₂O/CH₂Cl₂ (95:5, 0.5 L) and the aq. layer is saturated with salt. The organic phase is removed, the aq. phase is extracted with Et₂O/CH₂Cl₂ (95:5, 2×0.5 L) and the combined organic phases are dried (Na₂SO₄). Concentration in vacuo gives N-methyl R-1-(2-furyl)-2-aminoethanol (6.50 g, 96%) as a pale orange oil which solidifies at freezer (−20° C.) temperatures. This material is determined to be analytically pure and is utilized without further purification. Physical Characteristics:

¹H-NMR (400 MHz, DMSO-d₆): δ=7.55(m, 1), 6.37(m, 1), 6.25(d, J=3.2 Hz, 1), 4.59(m, 1), 2.70(m, 2), 2.25(s, 3).

¹³C-NMR (100 MHz, DMSO-d₆): δ=157.3, 141.9, 110.5, 105.9, 65.5, 56.5, 36.5. IR (neat): 3318 (s,b), 3116 (s,b), 2945 (s,b), 2853 (s,b), 2801, 2085 (b), 2019 (b), 1474, 1452, 1151, 1065, 1010, 884, 738, 600, cm⁻¹

MS (CI) m/z (rel. intensity): 159 (M+NH4⁺, 14), 142 (M+H, base), 126 (15), 124 (8), 112 (4), 74 (7), 69 (6), 61 (18).

KF Moisture: 0.83%.

Anal. Calcd for C₇H₁₁NO₂: C, 59.56; H, 7.85; N, 9.92. Found: C, 59.90; H, 7.83; N. 9.68

Specific Rotation [α^{D}₂₅]=32 (c 0.96, EtOH).

### Example 17

### N-Methyl R-1-(2-furyl)-2-aminoethanol PHA-728907

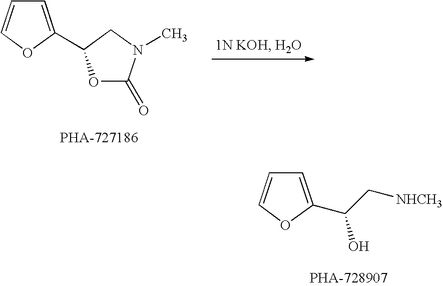

To PHA-727186 (8.0 g, 47.8 mmol) in a 500 mL 1N RB flask is added 1N aq. KOH (240 mL, 0.24 mol, 5 eq.). The flask is equipped with a reflux condenser, placed under nitrogen, then is immersed in a preheated (50° C.) oil bath. The mixture is allowed to stir and the PHA-727185 suspension slowly gave way to a clear solution. After stirring for 3 hours at 50° C. HPLC analysis indicated that the reaction is complete (HPLC: PHA-727188 RT=5.838 min, PHA-728907 RT=1.458 min). The mixture is cooled to room temperature and is cast into a separatory funnel, the flask is rinsed into the separatory funnel with Et₂O/CH₂Cl₂ (95:5, 0.5 L) and the aq. layer is saturated with salt. The organic phase is removed, the aq. phase is extracted with Et₂O/CH₂Cl₂ (95:5, 2×0.5 L) and the combined organic phases are dried (Na₂SO₄). Concentration in vacuo affords the desired aminoethanol PHA-728907(6.64 g, 98%) as a pale orange oil which solidifies at freezer (−20° C.) temperatures. This material is determined to be analytically pure and is utilized without further purification.

¹H-NMR (400 MHz, DMSO-d₆): δ=7.55(m, 1), 6.37(m, 1), 6.25(d, J=3.2 Hz, 1), 4.60(m, 1), 2.71(m, 2), 2.26(s, 3).

¹³C-NMR (100 MHz, DMSO-d₆): δ=157.3, 141.9, 110.5, 105.9, 65.5, 56.5, 36.2.

IR (neat): 3318 (s,b), 3116, 2945 (s,b), 2852 (s,b), 2801, 2086 (b), 2019 (b), 1474, 1453, 1151, 1065, 1010, 884, 738, 600, cm⁻¹

MS (CI) m/z (rel. intensity): 159 (M+NH4+, 2), 142 (M+H, base), 126 (14), 124 (18), 112 (2), 74 (2), 69 61 (10).

KF Moisture: 0.64%.

Anal. Calcd for C₇H₁₁NO₂: C, 59.56; H, 7.85; N, 9.92. Found: C, 59.28; H, 7.98; N, 9.80.

Specific Rotation [α^{D}₂₅]=−32 (c 0.91, EtOH).

The optical purities of the aminoethanols PHA728901 and PHA-728907 are difficult to determine by chiral HPLC due to non-baseline separation of the antipodes. Good analytical data is obtained by reconverting the aminoethanols to the related oxazolidinones with carbonyldiimidazole as shown below.

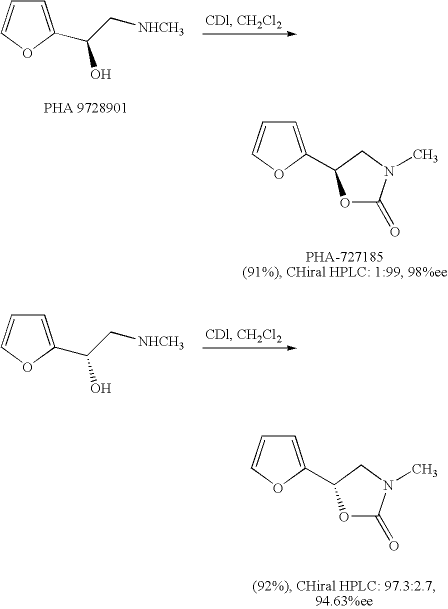

### Example 18

### Demonstration of Solvent Effect

Table 2 summarizes the results of reducing 3-chloroacetylpyridine. The reductions are conducted according to the procedure of Example 1 with the exception that solvent and pressure are varied as listed in the Table.

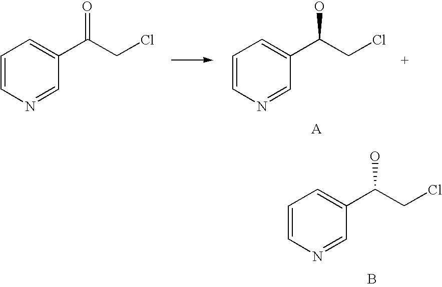

TABLE 2

| Et₃N/HCOOH + | | Overall | Ratio of | Pressure |
|---|---|---|---|---|
| Solvent | Time | Yield(%) | A/B | (mm Hg) |
| None | 48 h | 27 | 80/20 | atm |
| CH₂Cl₂ | 16 h | 39 | 85:15 | atm |
| THF | 16 h | 37 | 83:17 | atm |
| DMF | 16 h | 67 | 95/5 | atm |
| DMF | 0.75 h | 80 | 100/0 | 40 |

What is claimed is:

1. A method of preparing enantiomerically enriched amino alcohols of Formula I 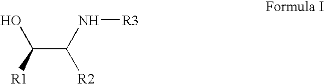
comprising:
a) reducing a carbonyl compound of Formula A 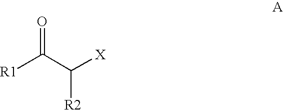
in a solvent in the presence of a reducing agent to give an alcohol of Formula B, 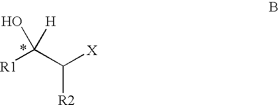
wherein R₁ is alkyl or heteroalkyl of 1-12 carbons, aryl or heteroaryl;
R₂ is H, alkyl of 1-4 carbons, CH₂-Aryl, or CH₂-heteroaryl; and
X is selected from the group Cl, Br, l, Aryl-SO₂O—, perfluoro alkyl-SO₂O— and alkyl-SO₂O—;
b) forming a urethane of Formula D from an alcohol of Formula B 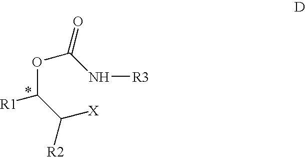
wherein R₃ is selected from the group alkyl of 1-6 carbons, aryl, benzyl, lower alkyl-CO, aryl-CO, lower alkyl-O—CO—, aryl-O—CO—, benzyl-O—CO— and aryl-SO₂—;
C) Forming an oxazolidinone of Formula E by treating a urethane of Formula D with a base; 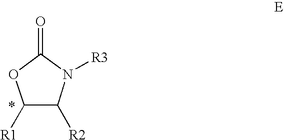
d) purifying an oxazolidinone of Formula E; and
e) converting an oxazolidinone of Formula E to an enantiomerically enriched amino alcohol of Formula 1.

2. The method of claim 1, wherein the reducing agent is a chiral catalyst.

3. The method of claim 2, wherein the chiral catalyst comprises ruthenium.

4. The method of claim 3, wherein the chiral catalyst is

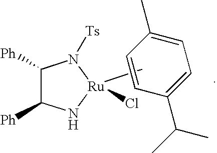

5. The method of claim 1, wherein the solvent in the reduction step A comprises DMF.

6. The method of claim 1, wherein the urethane of formula D is formed by reacting the alcohol of formula B with an isocyanate of Formula C; 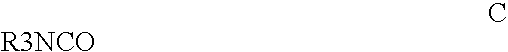
wherein R₃ is selected from the group alkyl of 1-6 carbons, aryl, benzyl, lower alkyl-CO, aryl-CO, lower alkyl-O—CO—, aryl-O—CO—, benzyl-O—CO— and aryl-SO₂—.

7. The method of claim 1, wherein the base used to form the oxazolidinone from the urethane of formula D comprises sodium hydride, potassium t-butoxide, sodium amylate, or sodium hydride.

8. The method of claim 1, wherein the enantiomerically enriched amino alcohol of formula I is greater than about 50% ee.

9. The method of claim 1, wherein the enantiomerically enriched amino alcohol of formula I is greater than about 80% ee.

10. The method of claim 1, wherein the enantiomerically enriched amino alcohol of formula I is greater than about 90% ee.

11. The method of claim 1, wherein the enantiomerically enriched amino alcohol of formula I is greater than about 95% ee.

12. The method of claim 1, wherein the enantiomerically enriched amino alcohol of formula I is greater than about 99% ee.

13. A method of preparing enantiomerically enriched amino alcohols of Formula I 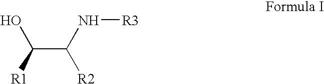
comprising:
a) forming an oxazolidinone of Formula E by treating a urethane of Formula D with a base; 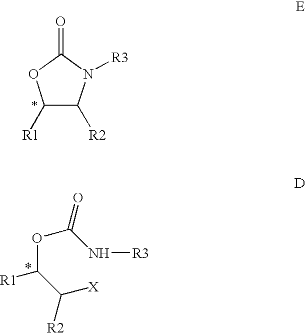
wherein:
R₁ is alkyl or heteroalkyl of 1-12 carbons, aryl or heteroaryl;
R₂ is H, alkyl of 1-4 carbons, CH₂-Aryl, or CH₂-heteroaryl;
R₃ is selected from the group alkyl of 1-6 carbons, aryl, benzyl, lower alkyl-CO, aryl-CO, lower alkyl-O—CO—, aryl-O—CO—, benzyl-O—CO— and aryl-SO₂—; and
X is selected from the group Cl, Br, I, Aryl-SO₂O—, perfluoro alkyl-SO₂O — and alkyl-SO₂O—; and
b) purifying the oxazolidinone of Formula E.

14. The method of claim 13, wherein the oxazolidinone is purified by recrystallization.

15. The method of claim 13 further comprising converting the oxazolidinone of Formula E to the enantiomerically enriched amino alcohol of Formula 1 by hydrolysis.

16. The method of claim 15, wherein the enantiomerically enriched amino alcohol of formula I is greater than about 50% ee.

17. The method of claim 16, wherein the enantiomerically enriched amino alcohol of formula I is greater than about 80% ee.

18. The method of claim 16, wherein the enantiomerically enriched amino alcohol of formula I is greater than about 90% ee.

19. The method of claim 16, wherein the enantiomerically enriched amino alcohol of formula I is greater than about 95% ee.

20. The method of claim 16, wherein the enantiomerically enriched amino alcohol of formula I is greater than about 99% ee.
